(19) United States
(12) Plant Patent Application Publication
(10) Pub. No.: US 2017/0354074 P1
(43) Pub. Date: Dec. 7, 2017

(54) Magnolia plant named 'Melissa Parris'

(50) ;

(71) Applicant: Kevin Parris, Spartanburg, SC (US)

(72) Inventors: Kevin Parris, Spartanburg, SC (US); Dennis Ledvina, (US)

(21) Appl. No.: 14/999,608

(22) Filed: Jun. 3, 2016

(51) Int. Cl. A01H 5/02

(52) U.S. Cl. CPC A01H 5/02
USPC PLT/223

(57) ABSTRACT

A new and distinct Magnolia cultivar named ‘Melissa Parris’ is disclosed, characterized by having pleasantly scented pink flowers, a strong central leader, and large tardily deciduous foliage. The new variety is a Magnolia, normally produced as a medium to large ornamental tree.

### BACKGROUND OF THE INVENTION

The present invention relates to a new and distinct cultivar of a deciduous Magnolia hybrid resulting from the intentional breeding of Magnolia [tripetala×(tripetala×obovata)] ‘Silk Road’ with Magnolia insignis MGA 355, now registered as the cultivar Magnolia insignis ‘Anita Figlar’. The objective of the breeding effort was to produce a large pink flowered magnolia cultivar that would flower after typical spring frosts. Pollen from harvested flowers of unpatented selection Magnolia insignis ‘Anita Figlar’ was isolated and dehisced by co-inventor Parris in May 2010, and subsequently used to hand pollinate flowers of the unpatented selection Magnolia hybrid ‘Silk Road’ by co-inventor Ledvina. A portion of the resulting seeds from the cross were carefully collected, and thereafter germinated and planted by co-inventor Parris in 2011. The resulting new variety was grown to flowering age in full sun, and first observed to flower in May 2014. The plant was thereafter selected for propagation by co-inventor Parris in May 2015 after observing prolific, ornamental flowering that commenced after the last average frost date in the upstate of South Carolina. The present exemplar stands 20 feet (6.0 m) tall by 8 feet (2.4 m) diameter. It has demonstrated excellent hardiness, having withstood Summer temperatures of 101° F. (38° C.) and Winter low temperatures of 6° F. (−14° C.).

Asexual reproduction of the cultivar ‘Melissa Parris’ has been repeatedly conducted under the direction of co-inventor Parris from vegetative cuttings in Gainesville, Ga. and Spartanburg, S.C. Small rooted cuttings from the novel plant have flowered and grown with vigor, thereby demonstrating that the cultivar is stable. Co-inventor Parris has also successfully reproduced the cultivar by chip budding using in-ground and container specimens of the hybrid Magnolia yuyuanensis×Magnolia insignis as rootstock.

### SUMMARY OF THE INVENTION

The cultivar ‘Melissa Parris’ has not been observed under all possible environmental conditions. The phenotype may vary somewhat with variations in temperature, day length, moisture availability, and light intensity, however no variance in genotype would result, as may be determined by genetic characterization, with methods such as inter-simple sequence repeat (ISSR) markers.

The following traits have been repeatedly observed and are determined to be the unique characteristics of Magnolia ‘Melissa Parris’. These characteristics in combination distinguish ‘Melissa Parris’ as a new and distinct cultivar.

1. ‘Melissa Parris’ exhibits hybrid vigor

2. ‘Melissa Parris’ exhibits a strong central leader with compact branch arrangement

3. ‘Melissa Parris’ exhibits large attractive glossy foliage

4. ‘Melissa Parris’ exhibits relatively large, lotus-like flowers

5. ‘Melissa Parris’ exhibits pink outer tepals having a color of RHS Red-Purple Group 65A

6. ‘Melissa Parris’ exhibits alternate pink and white striated inner tepals

7. ‘Melissa Parris’ produces a pleasant sweet citrus-like floral fragrance

8. ‘Melissa Parris’ exhibits late Spring to early Summer bloom

### PARENT COMPARISON

Plants of the new cultivar ‘Melissa Parris’ exhibit intermediate characteristics between that of the seed parent, Magnolia hybrid ‘Silk Road’, and that of the pollen parent Magnolia insignis ‘Anita Figlar’.

‘Melissa Parris’ retains some of the deciduous nature, large foliage, and large flowers of the seed parent, ‘Silk Road’, however the leaf drop is tardy in contrast, and the leaves have a darker, glossier appearance than the seed parent. The flowers of ‘Silk Road’ are white, while the flowers of new cultivar ‘Melissa Parris’ are smaller and distinctively pink with striations.

‘Melissa Parris’ vividly exhibits aspects of the pink/red floral pigmentation of the pollen parent, ‘Anita Figlar’ (RHS Red Group 53A), but in contrast produces deep pink outer tepals (RHS Red-Purple Group 65A) and alternating pink and white inner tepals. Inner tepals may be further distinguished from either parent by vein-like striations and blends of pink along a white background. ‘Melissa Parris’ flowers are markedly larger than the pollen parent (FIG. 4).

The seed and pollen parents are the best comparisons to the new cultivar ‘Melissa Parris’

### BRIEF DESCRIPTION OF THE PHOTOGRAPHS

The accompanying photographs Illustrate in full color, unique aspects of ‘Melissa Parris’.

FIG. 1, shows the entire plant at age 5 years;

FIG. 2, shows a fully open ‘Melissa Parris’ flower with a reference engineer's scale;

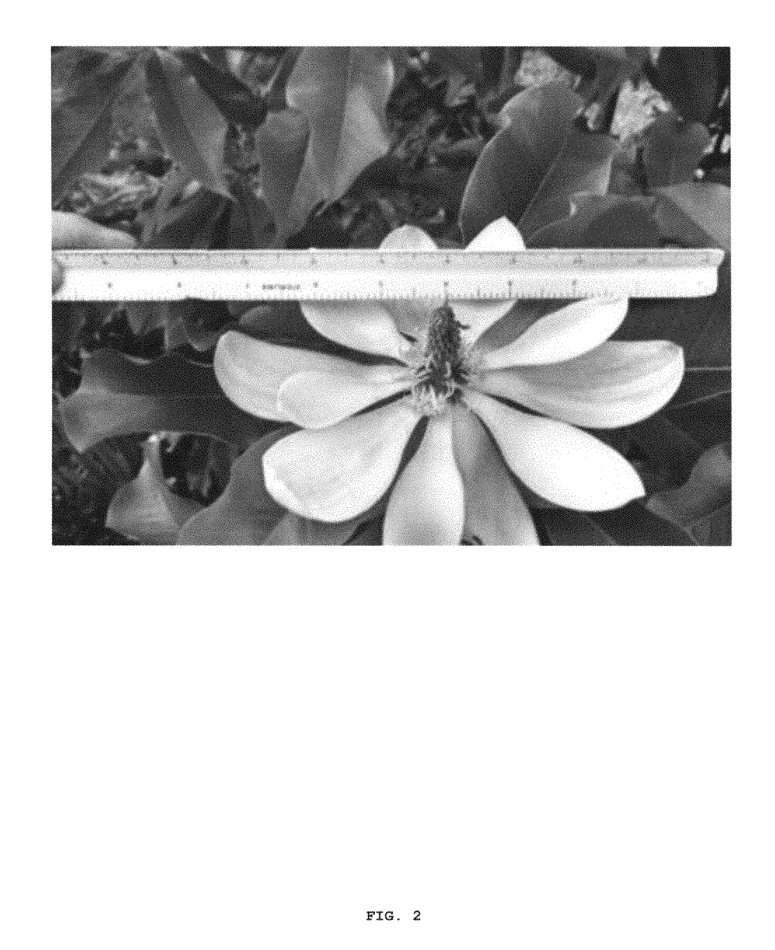

FIG. 3, shows a flower of ‘Melissa Parris’ with a reference R.H.S. Color Chart; and,

FIG. 4, shows a flower of ‘Melissa Parris’ on the left side with a flower of the pollen parent M. insignis ‘Anita Figlar’ to its right.

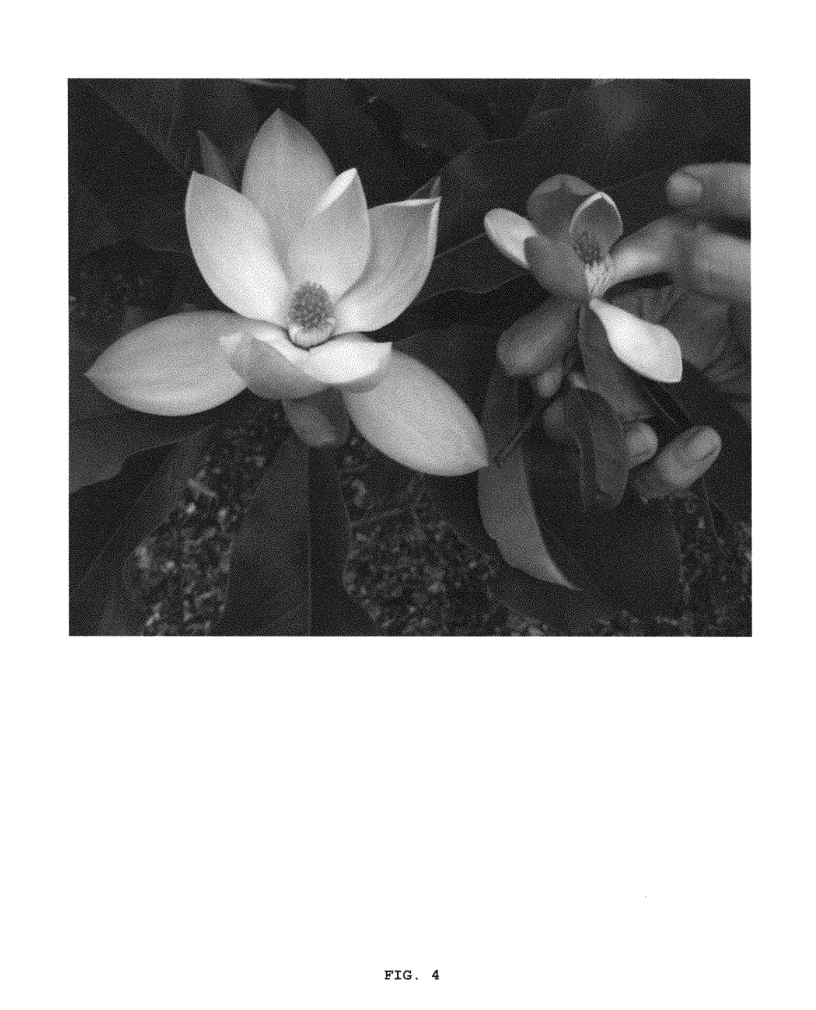

### DETAILED BOTANICAL DESCRIPTION

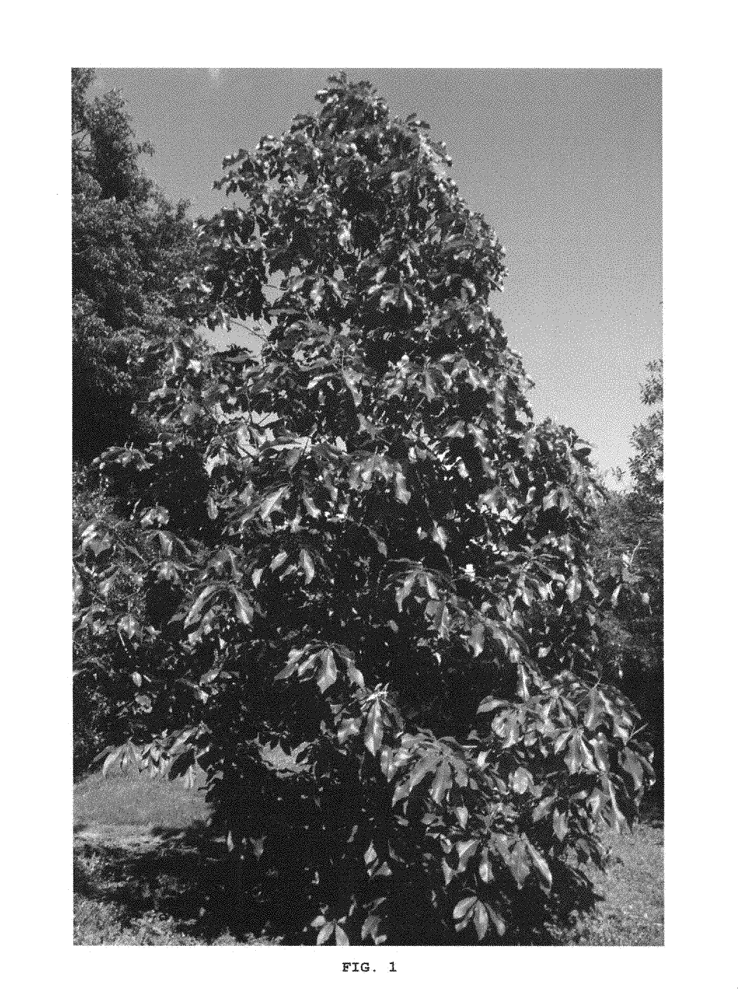

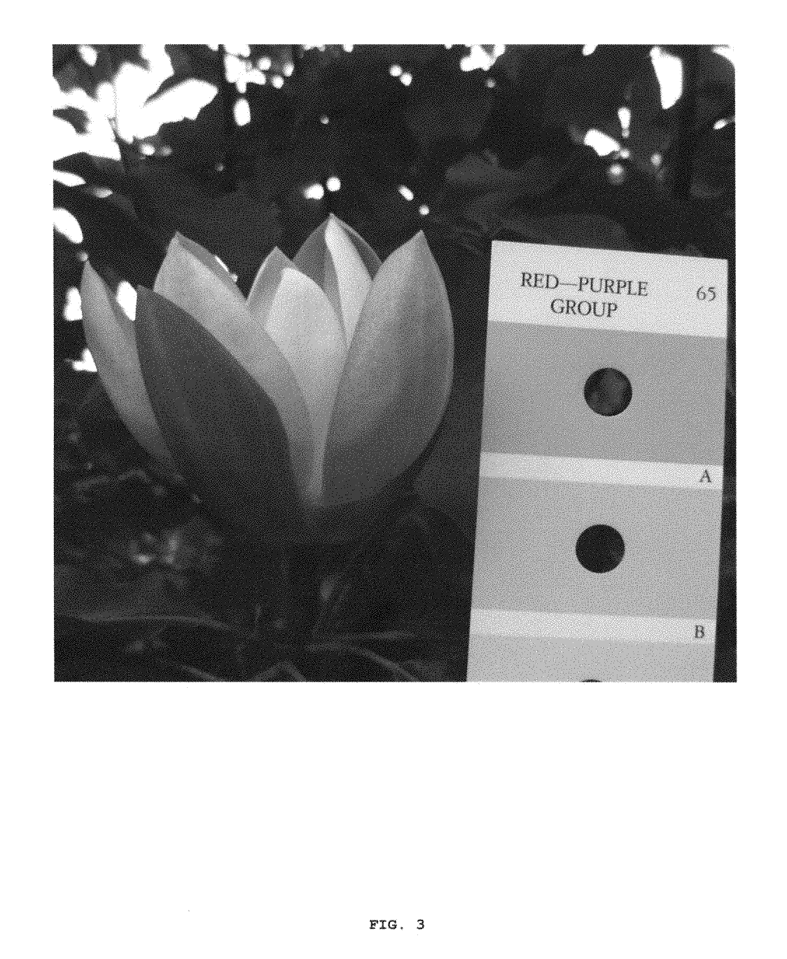

In the following description, color references are made to The Royal Horticulture Society Colour Chart except where general terms of ordinary dictionary significance are used. The following observations and measurements describe ‘Melissa Parris’ grown outdoors in Spartanburg, S.C., USDA Zone 7B. Measurements and numerical values represent averages of typical plant features.
Botanical classification: Magnolia ‘Melissa Parris’.
Propagation:
Time to initiate roots: About 40 days at approximately 30° C.
Time to produce a rooted cutting: About 90 days at approximately 30° C.; transplanted carefully following spring bud break at about 250 days.
Root description: Typical Magnolia root, thick, coarse, and white, aging to pale tan.
Time to chip bud: Spring budding proven successful on Magnolia yuyuanensis×Magnolia insignis root stock. 3-4 weeks from budding to bud break.
Plant:
   1. Growth habit.—Upright tree with strong central leader (FIG. 1).
   2. Age of plant described.—5 years.
   3. In container or in the ground.—Container 1 year, then ground 4 years.
   4. Height.—Approximately 6.0 meters.
   5. Spread.—Approximately 2.5 meters.
   6. Growth rate.—1.2 meters per year.
   7. Branching characteristics.—Single trunked tree, with ascending lateral branches forming at varying acute angles, 45-80 degrees to the trunk.
   8. Length of primary lateral branches.—2-3 meters
   9. Diameter of primary lateral branches.—Approximately 2 cm.
   10. Quantity of primary lateral branches.—46.
   11. Branch arrangement.—Alternate.
Trunk:
   1. Diameter.—7 centimeters after 5 years.
   2. Texture.—Moderately smooth with fine, sinuous, vertical striations.
   3. Color.—Grey-Brown with prominent pale grey lenticels.
Foliage:
Leaf:
   4. Arrangement.—Alternate.
   5. Quantity.—7-10 clustered tightly within 5-7 centimeters at terminal end of each growth flush.
   6. Average length.—Approximately 27 cm.
   7. Average width.—Approximately 9 cm.
   8. Shape of lamina.—Obovate.
   9. Apex.—Acuminate.
   10. Base.—Cuneate.
   11. Margin.—Entire.
   12. Texture of top surface.—Smooth.
   13. Texture of bottom surface.—Lightly pubescent.
   14. Leaf internode length.—Approximately 13 cm at mid-branch.
   15. Color.—Upper Surface, Near RHS Yellow-Green Group 147.
   16. Venation.—Pinnate.
   17. Petiole.—Stipular scar approximately 40% of overall length.
Flower:
   18. Natural flowering season.—Mid April-Early June in the Southeastern US.
   19. Flowering onset.—Parent tree first bloomed as a 3 year old seedling. Rooted cuttings have flowered within one year of production.
   20. Flower type and habit.—Terminal buds initiate flowers that most commonly are displayed singly, but occasionally a subtended companion bud emerges near the completion of flowering of the primary terminal bud.
   21. Flower longevity on the plant.—Flowers open the first time between 6:30 and 8:00 PM remaining open until early morning when the 6-7 (rarely 9) inner tepals move back into an overlapping position with the 3 outer tepals remaining entirely reflexed. Approximately 24 hours after the first opening, the inner tepals open again more fully than the previous night and shed their stamens. Flowers retain the blend of dark pink and creamy pink effectively into the 3^{rd} day without looking spent and discolored like many magnolias in the post male phase. The 2 days of tepal movements have been observed on cut branches in vases, making the new cultivar ‘Melissa Parris’ an ideal cut flower specimen.
   22. Quantity of flowers.—Profuse by 5^{th} year on parent tree.
   23. Flower size.—Diameter — 16-18 cm, while fully open in 2^{nd} evening, male phase Height — 16-18 cm, while closed in 2^{nd} day, post female phase.
   24. Pedicel.—Length — 3.5-4.0 cm. straight on terminal buds of ascending branches, curved upward on terminal buds of lateral branches. Diameter — Approximately 0.75 cm
   8. Tepals.—Color — Outer: RHS Red-Purple Group 65A (FIG. 3); Inner: White with stations and blends of RHS Red-Purple Group 65A Arrangement — Alternately whorled, overlapping. Shape — Spatulate — Obovate, Cupped Size — Length 8.0-9.0 cm, Width — 3.8-4.5 cm at widest point Margin — Entire Apex — Obtuse Tepal Quantity — 9-12, Commonly 10 Texture — Smooth.
   9. Fragrance.—Intense blend of sweet citrusy fragrances.
Reproductive organs:
Gynoecium: Approximately 1.25 (at widest point)×2.5 cm long Pistils — approximately 40. White prior to tepal opening, Orange-Pink when open.
Androecium: Approximately 0.6×0.6 cm Stamens — exceeding 100. Red at basal attachment, White from tip over ⅔ total length.
Other characteristics:
Seeds and fruits: The new cultivar has not proven to be self-fruitful, but fruit and viable seed has been produced from crosses with several Magnolia species.
Disease/pest resistance: Neither resistance nor susceptibility to pathogens or pests have been observed.

As herein used, the term “dehisce” refers to splitting or fracturing of an outer membrane to access an interior.

As herein used, the term “tepal” refers to outer flower parts of species having indistinguishable sepals and petals.

We claim:

1. A new and distinct hybrid of Magnolia ‘Silk Road’×Magnolia insignis ‘Anita Figlar’ named Magnolia ‘Melissa Parris’ as herein illustrated and described.
